(19) United States
(12) Patent Application Publication
(10) Pub. No.: US 2012/0109230 A1
(43) Pub. Date: May 3, 2012

(54) Neurostimulation system and method for graphically displaying electrode stimulation values

(75) Inventors: Sridhar Kothandaraman, Valencia, CA (US); Mun Pook Lui, Northridge, CA (US); Christopher Britton Gould, Valencia, CA (US)

(73) Assignee: BOSTON SCIENTIFIC NEUROMODULATION CORPORATION, Valencia, CA (US)

(21) Appl. No.: 13/200,629

(22) Filed: Sep. 26, 2011

Related U.S. Application Data
(60) Provisional application No. 61/409,905, filed on Nov. 3, 2010.

(51) Int. Cl. A61N 1/372

(52) U.S. Cl. 607/2

(57) ABSTRACT

An external control device for use with a neurostimulation system having a neurostimulation lead carrying a plurality of electrodes capable of conveying an electrical stimulation field into tissue in which the electrodes are implanted. The external control device comprises a user interface including one or more control elements and a display screen, and a processor configured for individually assigning stimulation amplitude values for selected ones of the electrodes in response to actuations of the one or more control elements and for displaying on the display screen representations of the electrodes and a plurality of first non-alphanumeric indicators of the stimulation amplitude values in graphical association with the respective representations of the selected electrodes.

### RELATED APPLICATION DATA

The present application claims the benefit under 35 U.S.C. §119 to U.S. provisional patent application Ser. No. 61/409,905, filed Nov. 3, 2010. The foregoing application is hereby incorporated by reference into the present application in its entirety.

### FIELD OF THE INVENTION

The present inventions relate to tissue stimulation systems, and more particularly, to neurostimulation systems for programming neurostimulation leads.

### BACKGROUND OF THE INVENTION

Implantable neurostimulation systems have proven therapeutic in a wide variety of diseases and disorders. Pacemakers and Implantable Cardiac Defibrillators (ICDs) have proven highly effective in the treatment of a number of cardiac conditions (e.g., arrhythmias). Spinal Cord Stimulation (SCS) systems have long been accepted as a therapeutic modality for the treatment of chronic pain syndromes, and the application of tissue stimulation has begun to expand to additional applications such as angina pectoralis and incontinence. Deep Brain Stimulation (DBS) has also been applied therapeutically for well over a decade for the treatment of refractory chronic pain syndromes, and DBS has also recently been applied in additional areas such as movement disorders and epilepsy. Further, in recent investigations, Peripheral Nerve Stimulation (PNS) systems have demonstrated efficacy in the treatment of chronic pain syndromes and incontinence, and a number of additional applications are currently under investigation. Furthermore, Functional Electrical Stimulation (FES) systems, such as the Freehand system by NeuroControl (Cleveland, Ohio), have been applied to restore some functionality to paralyzed extremities in spinal cord injury patients.

These implantable neurostimulation systems typically include one or more electrode carrying stimulation leads, which are implanted at the desired stimulation site, and a neurostimulator (e.g., an implantable pulse generator (IPG)) implanted remotely from the stimulation site, but coupled either directly to the stimulation lead(s) or indirectly to the stimulation lead(s) via a lead extension. The neurostimulation system may further comprise an external control device to remotely instruct the neurostimulator to generate electrical stimulation pulses in accordance with selected stimulation parameters.

Electrical stimulation energy may be delivered from the neurostimulator to the electrodes in the form of an electrical pulsed waveform. Thus, stimulation energy may be controllably delivered to the electrodes to stimulate neural tissue. The combination of electrodes used to deliver electrical pulses to the targeted tissue constitutes an electrode combination, with the electrodes capable of being selectively programmed to act as anodes (positive), cathodes (negative), or left off (zero). In other words, an electrode combination represents the polarity being positive, negative, or zero. Other parameters that may be controlled or varied include the amplitude, width, and rate of the electrical pulses provided through the electrode array. Each electrode combination, along with the electrical pulse parameters, can be referred to as a “stimulation parameter set.”

With some neurostimulation systems, and in particular, those with independently controlled current or voltage sources, the distribution of the current to the electrodes (including the case of the neurostimulator, which may act as an electrode) may be varied such that the current is supplied via numerous different electrode configurations. In different configurations, the electrodes may provide current or voltage in different relative percentages of positive and negative current or voltage to create different electrical current distributions (i.e., fractionalized electrode combinations).

As briefly discussed above, an external control device can be used to instruct the neurostimulator to generate electrical stimulation pulses in accordance with the selected stimulation parameters. Typically, the stimulation parameters programmed into the neurostimulator can be adjusted by manipulating controls on the external control device to modify the electrical stimulation provided by the neurostimulator system to the patient. Thus, in accordance with the stimulation parameters programmed by the external control device, electrical pulses can be delivered from the neurostimulator to the stimulation electrode(s) to stimulate or activate a volume of tissue in accordance with a set of stimulation parameters and provide the desired efficacious therapy to the patient. The best stimulus parameter set will typically be one that delivers stimulation energy to the volume of tissue that must be stimulated in order to provide the therapeutic benefit (e.g., treatment of pain), while minimizing the volume of non-target tissue that is stimulated.

However, the number of electrodes available, combined with the ability to generate a variety of complex stimulation pulses, presents a huge selection of stimulation parameter sets to the clinician or patient. For example, if the neurostimulation system to be programmed has an array of sixteen electrodes, millions of stimulation parameter sets may be available for programming into the neurostimulation system. Today, neurostimulation system may have up to thirty-two electrodes, thereby exponentially increasing the number of stimulation parameters sets available for programming.

To facilitate such selection, the clinician generally programs the neurostimulator through a computerized programming system. This programming system can be a self-contained hardware/software system, or can be defined predominantly by software running on a standard personal computer (PC). The PC or custom hardware may actively control the characteristics of the electrical stimulation generated by the neurostimulator to allow the optimum stimulation parameters to be determined based on patient feedback or other means and to subsequently program the neurostimulator with the optimum stimulation parameter set or sets, which will typically be those that stimulate all of the target tissue in order to provide the therapeutic benefit, yet minimizes the volume of non-target tissue that is stimulated. The computerized programming system may be operated by a clinician attending the patient in several scenarios.

For example, in order to achieve an effective result from SCS, the lead or leads must be placed in a location, such that the electrical stimulation will cause paresthesia. The paresthesia induced by the stimulation and perceived by the patient should be located in approximately the same place in the patient's body as the pain that is the target of treatment. If a lead is not correctly positioned, it is possible that the patient will receive little or no benefit from an implanted SCS system. Thus, correct lead placement can mean the difference between effective and ineffective pain therapy. When electrical leads are implanted within the patient, the computerized programming system, in the context of an operating room (OR) mapping procedure, may be used to instruct the neurostimulator to apply electrical stimulation to test placement of the leads and/or electrodes, thereby assuring that the leads and/or electrodes are implanted in effective locations within the patient.

Once the leads are correctly positioned, a fitting procedure, which may be referred to as a navigation session, may be performed using the computerized programming system to program the external control device, and if applicable the neurostimulator, with a set of stimulation parameters that best addresses the painful site. Thus, the navigation session may be used to pinpoint the stimulation region or areas correlating to the pain. Such programming ability is particularly advantageous for targeting the tissue during implantation, or after implantation should the leads gradually or unexpectedly move that would otherwise relocate the stimulation energy away from the target site. By reprogramming the neurostimulator (typically by independently varying the stimulation energy on the electrodes), the stimulation region can often be moved back to the effective pain site without having to re-operate on the patient in order to reposition the lead and its electrode array. When adjusting the stimulation region relative to the tissue, it is desirable to make small changes in the proportions of current, so that changes in the spatial recruitment of nerve fibers will be perceived by the patient as being smooth and continuous and to have incremental targeting capability.

One known computerized programming system for SCS is called the Bionic Navigator®, available from Boston Scientific Neuromodulation Corporation. The Bionic Navigator® is a software package that operates on a suitable PC and allows clinicians to program stimulation parameters into an external handheld programmer (referred to as a remote control). Each set of stimulation parameters, including fractionalized current distribution to the electrodes (as percentage cathodic current, percentage anodic current, or off), may be stored in both the Bionic Navigator® and the remote control and combined into a stimulation program that can then be used to stimulate multiple regions within the patient.

Prior to creating the stimulation programs, the Bionic Navigator® may be operated by a clinician in a “manual mode” to manually select the percentage cathodic current and percentage anodic current flowing through the electrodes, or may be operated by the clinician in an “automated mode” to electrically “steer” the current along the implanted leads in real-time (e.g., using a joystick or joystick-like controls), thereby allowing the clinician to determine the most efficacious stimulation parameter sets that can then be stored and eventually combined into stimulation programs.

Once a polarity and the amplitude (either as an absolute or a percentage) for the current or voltage on an active electrode is selected in a typical computerized programming system, the polarity and amplitude value may be alphanumerically displayed in association with this electrode to the user. However, due to the limited space on the display, it is sometimes difficult for the user to see the alphanumeric display of polarity/amplitude information in association with each active electrode, which problem is only worsened as the number of electrodes to be programmed increases (e.g., when the user interface must support sixteen or even thirty-two electrodes) and the display becomes more crowded as a result.

There, thus, remains a need to display polarity/amplitude information for active electrodes that can be more easily visualized by the user.

### SUMMARY OF THE INVENTION

In accordance with the present inventions, an external control device is provided. The external control device is for use with a neurostimulation system having a neurostimulation lead carrying a plurality of electrodes capable of conveying an electrical stimulation field into tissue in which the electrodes are implanted.

The external control device comprises a user interface including one or more control elements (e.g., graphical icons) and a display screen. The external control device further comprises a processor configured for individually assigning stimulation amplitude values for selected ones of the electrodes in response to actuations of the control elements(s) and for displaying on the display screen representations of the electrodes and a plurality of first non-alphanumeric indicators of the stimulation amplitude values in graphical association with the respective representations of the selected electrodes.

In one embodiment, the first non-alphanumeric indicators are different colors (chromatic or achromatic) for the respective stimulation amplitude values (e.g., different luminance of the same color hue (e.g., blue, red, gray, etc.)), different patterns or textures for the respective stimulation amplitude values, different partial fill objects for the respective stimulation amplitude values (e.g., different partially filed pie-shaped objects), etc. The first non-alphanumeric indicators may be graphically coupled to the representations of the electrodes corresponding to the selected electrodes. If the representations of the electrodes respectively take the form of closed geometric figures, the first non-alphanumeric indicators may be displayed in the closed geometric figures corresponding to the selected electrodes.

In another embodiment, the processor is further configured for individually assigning polarities for the selected ones of the electrodes in response to actuations of the one or more control elements, and for displaying second non-alphanumeric indicators of the polarities in direct graphical association with the respective representations of the selected electrodes. The second non-alphanumeric indicators may be, e.g., different color hues for the respective polarities. For example, a first of the different color hues may be blue for one of a positive polarity and a negative polarity, and a second of the different color hues may be red for the other of the positive polarity and the negative polarity.

In another embodiment, the control element(s) comprises a graphical control icon graphically coupled to the representation of one of the selected electrodes, and the processor is configured for increasing or decreasing the stimulation amplitude value for the selected electrode in response to actuation of the graphical control icon. In this case, the processor may be configured for displaying the first non-alphanumeric indicator of the increased or decreased stimulation amplitude value in association with the graphical control icon in addition to be displayed in association with the electrode representation. In still another embodiment, the control element(s) comprises a graphical palette that allows a user to discretely select one of a plurality of stimulation amplitude values for one of the selected electrodes, and processor is configured for assigning the one stimulation amplitude value, when selected by the user, to the selected electrode. In this case, the first non-alphanumeric indicators may be contained in the graphical palette in addition to be displayed in association with the electrode representations. In yet another embodiment, the control element(s) may comprise a graphical slider that allows a user to continuously select one of a plurality of stimulation amplitude values for one of the selected electrodes, and the processor is configured for assigning the one stimulation amplitude value, when selected by the user, to the selected electrode. In this case, the first non-alphanumeric indicators may be contained in the graphical slider in addition to be displayed in association with the electrode representations.

Other and further aspects and features of the invention will be evident from reading the following detailed description of the preferred embodiments, which are intended to illustrate, not limit, the invention.

### BRIEF DESCRIPTION OF THE DRAWINGS

The drawings illustrate the design and utility of preferred embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. The patent or application file contains at least one drawing executed in color. Copies of this patent or patent application publication with color drawing(s) will be provided by the Office upon request and payment of the necessary fee. Understanding that these drawings depict only typical embodiments of the invention and are not therefore to be considered limiting of its scope, the invention will be described and explained with additional specificity and detail through the use of the accompanying drawings in which:

FIG. 1 is a plan view of a Spinal cord Stimulation (SCS) system constructed in accordance with one embodiment of the present inventions;

FIG. 2 is a perspective view of the arrangement of the SCS system of FIG. 1 with respect to a patient;

FIG. 3 is a profile view of an implantable pulse generator (IPG) and percutaneous leads used in the SCS system of FIG. 1;

FIG. 4 is front view of a remote control (RC) used in the SCS system of FIG. 1;

FIG. 5 is a block diagram of the internal components of the RC of FIG. 4;

FIG. 6 is a block diagram of the internal components of a clinician's programmer (CP) used in the SCS system of FIG. 1;

FIGS. 7a and 7b are plan views of a user interface of the CP of FIG. 6 for programming the IPG of FIG. 3;

FIGS. 8a-8n are plan views respectively illustrating the different polarity and stimulation amplitude value indicators for the electrodes displayed in the user interface of FIGS. 7a and 7b.

### DETAILED DESCRIPTION OF THE EMBODIMENTS

The description that follows relates to a spinal cord stimulation (SCS) system. However, it is to be understood that while the invention lends itself well to applications in SCS, the invention, in its broadest aspects, may not be so limited. Rather, the invention may be used with any type of implantable electrical circuitry used to stimulate tissue. For example, the present invention may be used as part of a pacemaker, a defibrillator, a cochlear stimulator, a retinal stimulator, a stimulator configured to produce coordinated limb movement, a cortical stimulator, a deep brain stimulator, peripheral nerve stimulator, microstimulator, or in any other neurostimulator configured to treat urinary incontinence, sleep apnea, shoulder sublaxation, headache, etc.

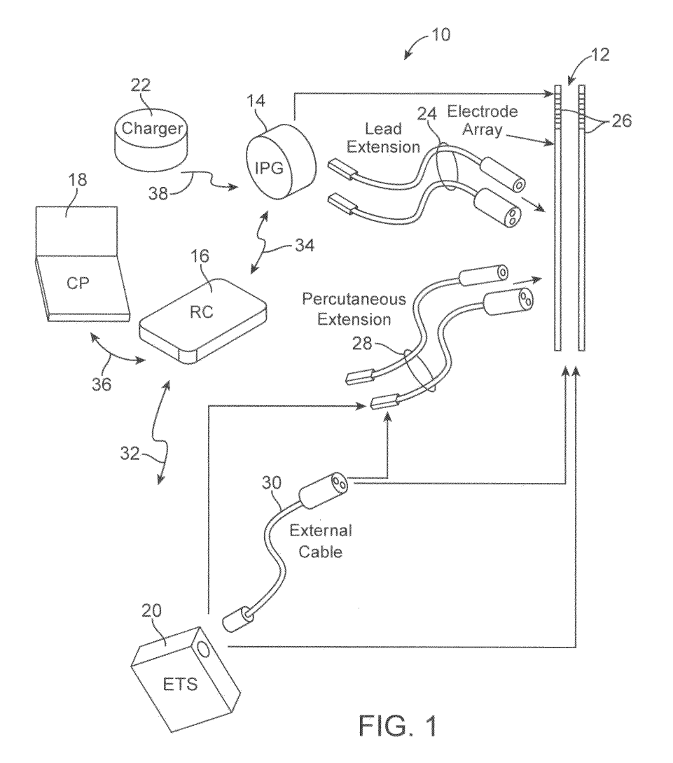

Turning first to FIG. 1, an exemplary SCS system 10 generally includes a plurality (in this case, two) of implantable neurostimulation leads 12, an implantable pulse generator (IPG) 14, an external remote controller RC 16, a clinician's programmer (CP) 18, an external trial stimulator (ETS) 20, and an external charger 22.

The IPG 14 is physically connected via one or more percutaneous lead extensions 24 to the neurostimulation leads 12, which carry a plurality of electrodes 26 arranged in an array. In the illustrated embodiment, the neurostimulation leads 12 are percutaneous leads, and to this end, the electrodes 26 are arranged in-line along the neurostimulation leads 12. As will be described in further detail below, the IPG 14 includes pulse generation circuitry that delivers electrical stimulation energy in the form of a pulsed electrical waveform (i.e., a temporal series of electrical pulses) to the electrode array 26 in accordance with a set of stimulation parameters.

The ETS 20 may also be physically connected via the percutaneous lead extensions 28 and external cable 30 to the neurostimulation leads 12. The ETS 20, which has similar pulse generation circuitry as the IPG 14, also delivers electrical stimulation energy in the form of a pulse electrical waveform to the electrode array 26 accordance with a set of stimulation parameters. The major difference between the ETS 20 and the IPG 14 is that the ETS 20 is a non-implantable device that is used on a trial basis after the neurostimulation leads 12 have been implanted and prior to implantation of the IPG 14, to test the responsiveness of the stimulation that is to be provided. Thus, any functions described herein with respect to the IPG 14 can likewise be performed with respect to the ETS 20. Further details of an exemplary ETS are described in U.S. Pat. No. 6,895,280, which is expressly incorporated herein by reference.

The RC 16 may be used to telemetrically control the ETS 20 via a bi-directional RF communications link 32. Once the IPG 14 and neurostimulation leads 12 are implanted, the RC 16 may be used to telemetrically control the IPG 14 via a bi-directional RF communications link 34. Such control allows the IPG 14 to be turned on or off and to be programmed with different stimulation parameter sets. The IPG 14 may also be operated to modify the programmed stimulation parameters to actively control the characteristics of the electrical stimulation energy output by the IPG 14. As will be described in further detail below, the CP 18 provides clinician detailed stimulation parameters for programming the IPG 14 and ETS 20 in the operating room and in follow-up sessions.

The CP 18 may perform this function by indirectly communicating with the IPG 14 or ETS 20, through the RC 16, via an IR communications link 36. Alternatively, the CP 18 may directly communicate with the IPG 14 or ETS 20 via an RF communications link (not shown). The clinician detailed stimulation parameters provided by the CP 18 are also used to program the RC 16, so that the stimulation parameters can be subsequently modified by operation of the RC 16 in a stand-alone mode (i.e., without the assistance of the CP 18).

The external charger 22 is a portable device used to transcutaneously charge the IPG 14 via an inductive link 38. For purposes of brevity, the details of the external charger 22 will not be described herein. Details of exemplary embodiments of external chargers are disclosed in U.S. Pat. No. 6,895,280, which has been previously incorporated herein by reference. Once the IPG 14 has been programmed, and its power source has been charged by the external charger 22 or otherwise replenished, the IPG 14 may function as programmed without the RC 16 or CP 18 being present.

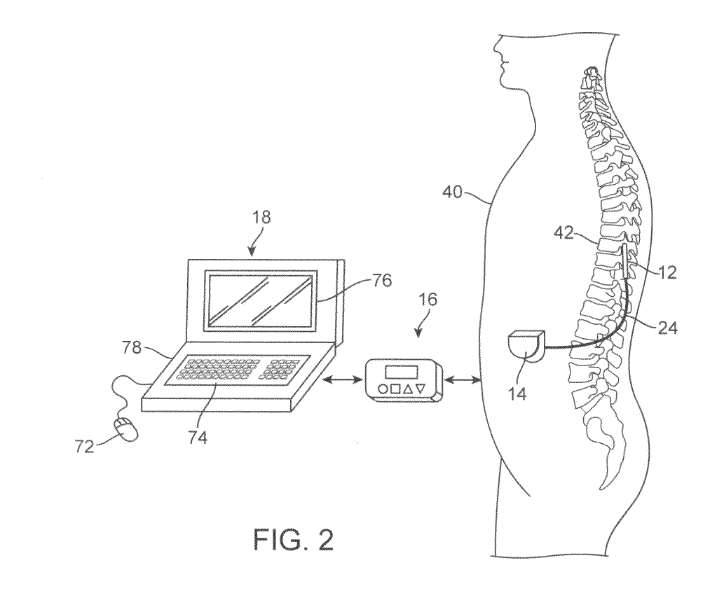

As shown in FIG. 2, the electrode leads 12 are implanted within the spinal column 42 of a patient 40. The preferred placement of the electrode leads 12 is adjacent, i.e., resting upon, the spinal cord area to be stimulated. Due to the lack of space near the location where the electrode leads 12 exit the spinal column 42, the IPG 14 is generally implanted in a surgically-made pocket either in the abdomen or above the buttocks. The IPG 14 may, of course, also be implanted in other locations of the patient's body. The lead extensions 24 facilitate locating the IPG 14 away from the exit point of the electrode leads 12. As there shown, the CP 18 communicates with the IPG 14 via the RC 16.

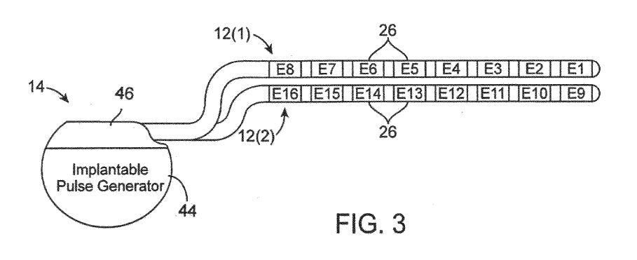

Referring now to FIG. 3, the external features of the neurostimulation leads 12 and the IPG 14 will be briefly described. One of the neurostimulation leads 12(1) has eight electrodes 26 (labeled E1-E8), and the other neurostimulation lead 12(2) has eight electrodes 26 (labeled E9-E16). The actual number and shape of leads and electrodes will, of course, vary according to the intended application. The IPG 14 comprises an outer housing 44 for housing the electronic and other components (described in further detail below), and a connector 46 to which the proximal ends of the neurostimulation leads 12 mates in a manner that electrically couples the electrodes 26 to the electronics within the outer housing 44. The outer housing 44 is composed of an electrically conductive, biocompatible material, such as titanium, and forms a hermetically sealed compartment wherein the internal electronics are protected from the body tissue and fluids. In some cases, the outer housing 44 may serve as an electrode.

The IPG 14 includes a battery and pulse generation circuitry that delivers the electrical stimulation energy in the form of a pulsed electrical waveform to the electrode array 26 in accordance with a set of stimulation parameters programmed into the IPG 14. Such stimulation parameters may comprise electrode combinations, which define the electrodes that are activated as anodes (positive), cathodes (negative), and turned off (zero), percentage of stimulation energy assigned to each electrode (fractionalized electrode combinations), and electrical pulse parameters, which define the pulse amplitude (measured in milliamps or volts depending on whether the IPG 14 supplies constant current or constant voltage to the electrode array 26), pulse width (measured in microseconds), and pulse rate (measured in pulses per second).

Electrical stimulation will occur between two (or more) activated electrodes, one of which may be the IPG case. Simulation energy may be transmitted to the tissue in a monopolar or multipolar (e.g., bipolar, tripolar, etc.) fashion. Monopolar stimulation occurs when a selected one of the lead electrodes 26 is activated along with the housing 44 of the IPG 14, so that stimulation energy is transmitted between the selected electrode 26 and case. Bipolar stimulation occurs when two of the lead electrodes 26 are activated as anode and cathode, so that stimulation energy is transmitted between the selected electrodes 26. For example, electrode E3 on the first lead 12 may be activated as an anode at the same time that electrode E11 on the second lead 12 is activated as a cathode. Tripolar stimulation occurs when three of the lead electrodes 26 are activated, two as anodes and the remaining one as a cathode, or two as cathodes and the remaining one as an anode. For example, electrodes E4 and E5 on the first lead 12 may be activated as anodes at the same time that electrode E12 on the second lead 12 is activated as a cathode.

In the illustrated embodiment, IPG 14 can individually control the magnitude of electrical current flowing through each of the electrodes. In this case, it is preferred to have a current generator, wherein individual current-regulated amplitudes from independent current sources for each electrode may be selectively generated. Although this system is optimal to take advantage of the invention, other stimulators that may be used with the invention include stimulators having voltage regulated outputs. While individually programmable electrode amplitudes are optimal to achieve fine control, a single output source switched across electrodes may also be used, although with less fine control in programming. Mixed current and voltage regulated devices may also be used with the invention. Further details discussing the detailed structure and function of IPGs are described more fully in U.S. Pat. Nos. 6,516,227 and 6,993,384, which are expressly incorporated herein by reference.

It should be noted that rather than an IPG, the SCS system 10 may alternatively utilize an implantable receiver-stimulator (not shown) connected to the neurostimulation leads 12. In this case, the power source, e.g., a battery, for powering the implanted receiver, as well as control circuitry to command the receiver-stimulator, will be contained in an external controller inductively coupled to the receiver-stimulator via an electromagnetic link. Data/power signals are transcutaneously coupled from a cable-connected transmission coil placed over the implanted receiver-stimulator. The implanted receiver-stimulator receives the signal and generates the stimulation in accordance with the control signals.

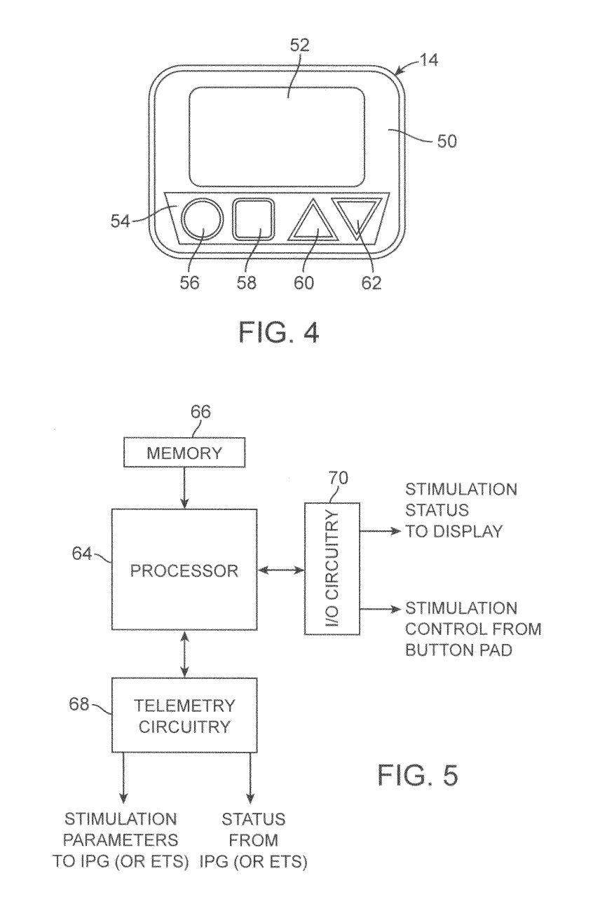

Referring now to FIG. 4, one exemplary embodiment of an RC 16 will now be described. As previously discussed, the RC 16 is capable of communicating with the IPG 14, CP 18, or ETS 20. The RC 16 comprises a housing 50, which houses internal componentry (including a printed circuit board (PCB)), and a lighted display screen 52 and button pad 54 carried by the exterior of the housing 50. In the illustrated embodiment, the display screen 52 is a lighted flat panel display screen, and the button pad 54 comprises a membrane switch with metal domes positioned over a flex circuit, and a keypad connector connected directly to a PCB. In an optional embodiment, the display screen 52 has touch screen capabilities. The button pad 54 includes a multitude of buttons 56, 58, 60, and 62, which allow the IPG 14 to be turned ON and OFF, provide for the adjustment or setting of stimulation parameters within the IPG 14, and provide for selection between screens.

In the illustrated embodiment, the button 56 serves as an ON/OFF button that can be actuated to turn the IPG 14 ON and OFF. The button 58 serves as a select button that allows the RC 16 to switch between screen displays and/or parameters. The buttons 60 and 62 serve as up/down buttons that can be actuated to increment or decrement any of stimulation parameters of the pulse generated by the IPG 14, including pulse amplitude, pulse width, and pulse rate. For example, the selection button 58 can be actuated to place the RC 16 in a “Pulse Amplitude Adjustment Mode,” during which the pulse amplitude can be adjusted via the up/down buttons 60, 62, a “Pulse Width Adjustment Mode,” during which the pulse width can be adjusted via the up/down buttons 60, 62, and a “Pulse Rate Adjustment Mode,” during which the pulse rate can be adjusted via the up/down buttons 60, 62. Alternatively, dedicated up/down buttons can be provided for each stimulation parameter. Rather than using up/down buttons, any other type of actuator, such as a dial, slider bar, or keypad, can be used to increment or decrement the stimulation parameters. Further details of the functionality and internal componentry of the RC 16 are disclosed in U.S. Pat. No. 6,895,280, which has previously been incorporated herein by reference.

Referring to FIG. 5, the internal components of an exemplary RC 16 will now be described. The RC 16 generally includes a processor 64 (e.g., a microcontroller), memory 66 that stores an operating program for execution by the processor 64, as well as stimulation parameter sets in a navigation table (described below), input/output circuitry, and in particular, telemetry circuitry 68 for outputting stimulation parameters to the IPG 14 and receiving status information from the IPG 14, and input/output circuitry 70 for receiving stimulation control signals from the button pad 54 and transmitting status information to the display screen 52 (shown in FIG. 4). As well as controlling other functions of the RC 16, which will not be described herein for purposes of brevity, the processor 64 generates new stimulation parameter sets in response to the user operation of the button pad 54. These new stimulation parameter sets would then be transmitted to the IPG 14 via the telemetry circuitry 68. Further details of the functionality and internal componentry of the RC 16 are disclosed in U.S. Pat. No. 6,895,280, which has previously been incorporated herein by reference.

As briefly discussed above, the CP 18 greatly simplifies the programming of multiple electrode combinations, allowing the user (e.g., the physician or clinician) to readily determine the desired stimulation parameters to be programmed into the IPG 14, as well as the RC 16. Thus, modification of the stimulation parameters in the programmable memory of the IPG 14 after implantation is performed by a user using the CP 18, which can directly communicate with the IPG 14 or indirectly communicate with the IPG 14 via the RC 16. That is, the CP 18 can be used by the user to modify operating parameters of the electrode array 26 near the spinal cord.

As shown in FIG. 2, the overall appearance of the CP 18 is that of a laptop personal computer (PC), and in fact, may be implemented using a PC that has been appropriately configured to include a directional-programming device and programmed to perform the functions described herein. Thus, the programming methodologies can be performed by executing software instructions contained within the CP 18. Alternatively, such programming methodologies can be performed using firmware or hardware. In any event, the CP 18 may actively control the characteristics of the electrical stimulation generated by the IPG 14 to allow the optimum stimulation parameters to be determined based on patient feedback and for subsequently programming the IPG 14 with the optimum stimulation parameters.

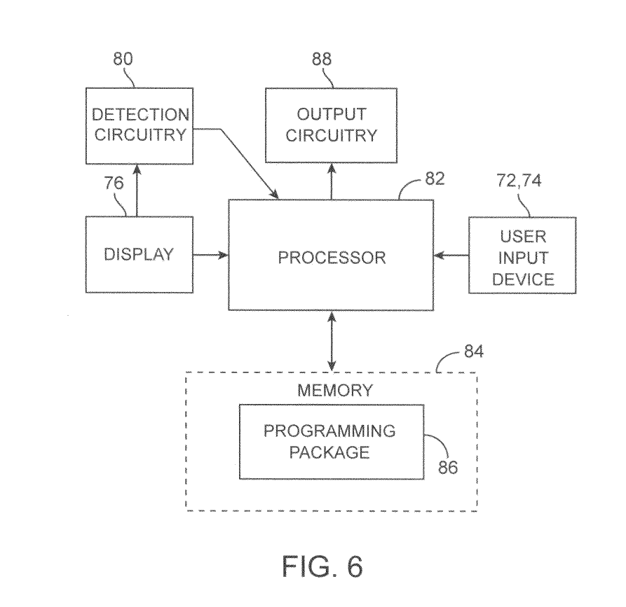

To allow the user to perform these functions, the CP 18 includes a mouse 72, a keyboard 74, and a programming display screen 76 housed in a housing 78. It is to be understood that in addition to, or in lieu of, the mouse 72, other directional programming devices may be used, such as a joystick, a button pad, a group of keyboard arrow keys, a roller ball tracking device, and horizontal and vertical rocker-type arm switches. Referring to FIG. 6, the CP 18 further includes detection circuitry 80 capable of detecting an actuation event on the display screen 76. Such actuation event may include placing at least one pointing element (not shown) in proximity to at least one graphical object displayed on the display screen 76, as well as possibly other events involving the point element(s), such as moving the pointing element(s) across the screen or clicking or tapping with the pointing element(s), as will be described in further detail below.

In the preferred embodiments described below, the display screen 76 takes the form of a digitizer touch screen, which may either passive or active. If passive, the display screen 76 includes detection circuitry that recognizes pressure or a change in an electrical current when a passive device, such as a finger or non-electronic stylus, contacts the screen. If active, the display screen 76 includes detection circuitry that recognizes a signal transmitted by an electronic pen or stylus. In either case, the detection circuitry 80 is capable of detecting when a physical pointing device (e.g., a finger, a non-electronic stylus, or an electronic stylus) is in close proximity to the screen, whether it be making physical contact between the pointing device and the screen or bringing the pointing device in proximity to the screen within a predetermined distance, as well as detecting the location of the screen in which the physical pointing device is in close proximity. When the pointing device touches or otherwise is in close proximity to the screen, the graphical object on the screen adjacent to the touch point is “locked” for manipulation, and when the pointing device is moved away from the screen the previously locked object is unlocked.

In some embodiments, the display screen 76 takes the form of a conventional screen, in which case, the pointing element is not an actual pointing device like a finger or stylus, but rather is a virtual pointing device, such as a cursor controlled by a mouse, joy stick, trackball, etc.

As shown in FIG. 6, the CP 18 generally further includes a processor 82 (e.g., a central processor unit (CPU)) and memory 84 that stores a stimulation programming package 86, which can be executed by the processor 82 to allow the user to program the IPG 14, and RC 16. The CP 18 further includes output circuitry 88 (e.g., via the telemetry circuitry of the RC 16) for downloading stimulation parameters to the IPG 14 and RC 16 and for uploading stimulation parameters already stored in the memory 66 of the RC 16, via the telemetry circuitry 68 of the RC 16.

Execution of the programming package 86 by the processor 82 provides a multitude of display screens (not shown) that can be navigated through via use of afore-described pointing device. These display screens allow the clinician to, among other functions, to select or enter patient profile information (e.g., name, birth date, patient identification, physician, diagnosis, and address), enter procedure information (e.g., programming/follow-up, implant trial system, implant IPG, implant IPG and lead(s), replace IPG, replace IPG and leads, replace or revise leads, explant, etc.), generate a pain map of the patient, define the configuration and orientation of the leads, initiate and control the electrical stimulation energy output by the leads 12, and select and program the IPG 14 with stimulation parameters in both a surgical setting and a clinical setting. Further details discussing the above-described CP functions are disclosed in U.S. patent application Ser. No. 12/501,282, entitled “System and Method for Converting Tissue Stimulation Programs in a Format Usable by an Electrical Current Steering Navigator,” and U.S. patent application Ser. No. 12/614,942, entitled “System and Method for Determining Appropriate Steering Tables for Distributing Stimulation Energy Among Multiple Neurostimulation Electrodes,” which are expressly incorporated herein by reference.

Most pertinent to the present inventions, execution of the programming package 86 provides a more intuitive user interface that allows a user to more easily visualize the current polarities and stimulation amplitude values for each of the electrodes 26 when programming the IPG 14.

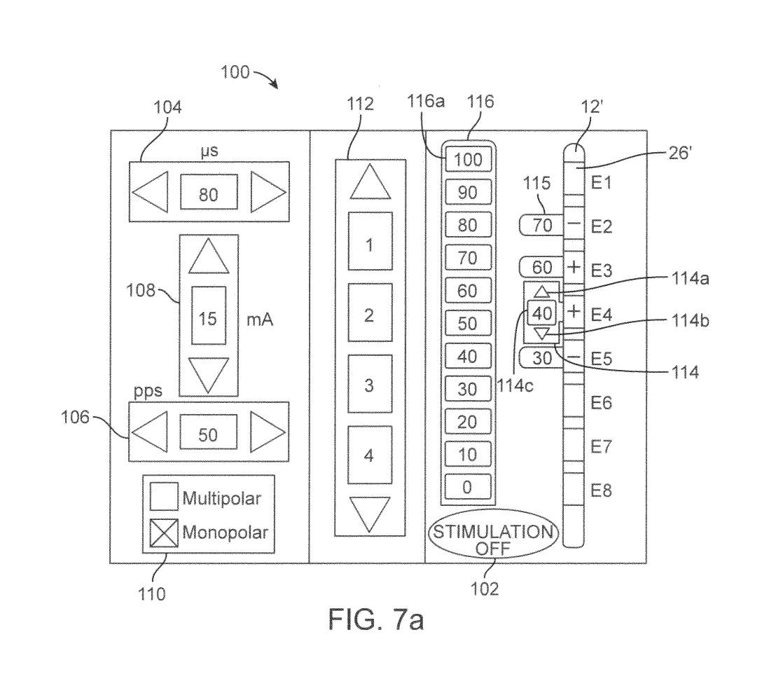

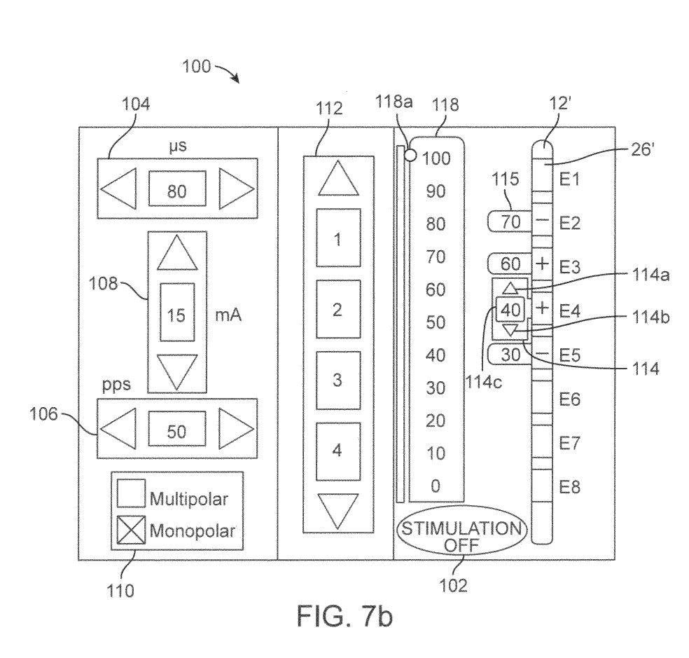

Referring now to FIGS. 7a and 7b, an exemplary programming screen 100 generated by the CP 16 to allow a user to program the IPG 14 will now be described. The programming screen 100 includes various control elements described below that can be actuated to perform various control functions.

A pointing element may be placed on any of the control elements to perform the actuation event. As described above, in the case of a digitizer touch screen, the pointing element will be an actual pointing element (e.g., a finger or active or passive stylus) that can be used to physically tap the screen above the respective graphical control element or otherwise brought into proximity with respect to the graphical control element. In the case of a conventional screen, the pointing element will be a virtual pointing element (e.g., a cursor) that can be used to graphically click on the respective control element.

The programming screen 100 comprises a stimulation on/off control 102 that can be alternately actuated initiate or cease the delivery of electrical stimulation energy from the IPG 14. The programming screen 100 further includes various stimulation parameter controls that can be operated by the user to manually adjust stimulation parameters. In particular, the programming screen 100 includes a pulse width adjustment control 104 (expressed in microseconds (μs)), a pulse rate adjustment control 106 (expressed in Hertz (Hz)), and a pulse amplitude adjustment control 108 (expressed in milliamperes (mA)). Each control includes a first arrow that can be actuated to decrease the value of the respective stimulation parameter and a second arrow that can be actuated to increase the value of the respective stimulation parameter. The programming screen 100 also includes multipolar/monopolar stimulation selection control 110, which includes check boxes that can be alternately actuated by the user to selectively provide multipolar or monopolar stimulation. The programming screen 100 also includes an electrode combination control 112 having arrows that can be actuated by the user to select one of four different electrode combinations 1-4. Each of the electrode combinations 1-4 can be created using various ones of the control elements.

The programming screen 100 displays graphical representations of the leads 12′ including the electrodes 26′. Significantly, the programming screen 100 includes graphical control elements (described in further detail below), the actuation of which will prompt the processor 82 to individually assign polarities (either, positive, negative, or off) and stimulation amplitude values for selected ones of the electrodes 26 and displaying indicators of the polarities and stimulation amplitude values in direct graphical association with the respective representations of the selected electrodes 26′. In the illustrated embodiments, the stimulation amplitude values are fractionalized electrical current values (% current), such that the stimulation amplitude values for each polarization totals to 100. However, in alternative embodiments, the stimulation amplitude values may be normalized current or voltage values (e.g., 1-10), absolute current or voltage values (e.g., mA or V), etc. Furthermore, the stimulation amplitude values may be parameters that are a function of current or voltage, such as charge (current amplitude×pulse width) or charge injected per second (current amplitude×pulse width×rate (or period)).

For the purposes of this specification, an indicator is in graphical association with an electrode representation 26′ if it is adjacent to and closer to that electrode representation 26′ than any other electrode representation 26′ in a manner that allows the user to recognize that the indicator provides information related to the electrode 26 corresponding to that electrode representation 26′. An indicator is in direct association with an electrode representation 26′ if is graphically within or graphically touches that electrode representation 26′.

In the illustrated embodiment, each electrode representation 26′ takes the form of a closed geometric figure, and in this case a rectangle, that can be touched or otherwise clicked to toggle the corresponding active electrode 26 between a positive polarity, a negative polarity, and an off-state. In alternative embodiments, the electrode representations 26′ can take the form of other types of closed geometric figures, such as circles. In essence, the electrode representations 26′ themselves operate as the graphical control elements the actuations of which prompt the processor 82 to assign the polarities to the selected electrodes 26. In alternative embodiments, control elements separate from the electrode representations 26′ may be used to change the polarity of the selected electrodes 26.

In any event, the programming screen 100 includes an alphanumeric indicator (i.e., letters, numbers, punctuation marks, and mathematical symbols) of the polarity of each of the selected electrodes 26. For example, in the illustrated embodiment, the alphanumeric indicators takes the form of a “+” representing a positive polarity (anode) and a “−” representing a negative polarity (cathode), each of which is displayed within the selected electrode representations 26′. In alternative embodiments, the alphanumeric indicators for the polarities are displayed adjacent to, but not inside, the corresponding electrode representations 26′. In either event, such polarity indicators are preferably displayed in graphical association with the respective electrode representations 26′.

The programming screen 100 includes a stimulation amplitude adjustment control 114 that appears next to the electrode representation 26′ that has been touched or clicked to prompt the processor 82 to change the polarity of the corresponding electrode 26. The stimulation amplitude adjustment control 114 includes an upper arrow 114a that can be actuated to increase the value of the stimulation amplitude assigned to the selected electrode 26, and a lower arrow 114b that can be actuated to decrease the value of the stimulation amplitude assigned to the selected electrode 26 (e.g., in 1% increments). The stimulation amplitude adjustment control 114 also includes an indicator 114c that provides an alphanumeric indication of the stimulation amplitude currently assigned to the selected electrode 26.

The programming screen 100 includes an alphanumeric indicator of the stimulation amplitude value previously assigned to each of the selected electrodes 26. For example, in the illustrated embodiment, the alphanumeric indicators takes the form of numbers (e.g., 10, 20, 30, etc.) representing the stimulation amplitude (and in the illustrated embodiment, the percentage of the total current) for the selected electrode 26. Each alphanumeric indicator is displayed in a “flag” 115 that is in direct graphical association with the selected electrode representations 26′.

As shown in FIG. 7a, the programming screen 100 further includes a stimulation amplitude palette 116 that allows a user to discretely select one of the stimulation amplitude values for a currently selected electrode 26, which will prompt the processor 82 to assign the selected stimulation amplitude value to the selected electrode 26. The stimulation amplitude palette 116 includes regions or areas 116a corresponding to the various discrete stimulation amplitude values, which regions or areas 116a can be individually actuated (with a physical pointing device or virtual pointing device) to select the respective stimulation amplitude value. In the illustrated embodiment, alphanumeric indicators of the stimulation amplitude values (i.e., 10%, 20%, 30%, etc.) are displayed in the respective palette regions 116a to allow the user to more easily correlate the regions 116a with the desired stimulation amplitude values. The stimulation amplitude adjustment control 114 and stimulation amplitude palette 116 can be used together in that the control 114 allows the user to finely adjust the stimulation amplitude values (e.g., by one percent increments), whereas the palette 116 allows the user to grossly adjust the stimulation amplitude values (e.g., by ten percent increments).

As shown in FIG. 7b, the programming screen 100 alternatively includes a stimulation amplitude slider 118 that allows a user to continually select one of the stimulation amplitude values for a currently selected electrode 26, which will prompt the processor 82 to assign the selected stimulation amplitude value to the selected electrode 26. The slider 118 includes a slider object 118a (in this case, a graphical ball) that can be slid (with a physical pointing device or virtual pointing device) to select the respective stimulation amplitude value. In the illustrated embodiment, the slider 118 is graduated with stimulation amplitude values (i.e., 10%, 20%, 30%, etc) to allow the user to more easily correlate the regions 116′ with the desired stimulation amplitude values, although in this case, stimulation amplitude values can be selected between increments of 10% (e.g., at 1% increments).

Significantly, the programming screen 100 further includes non-alphanumeric indicators of the polarities and stimulation amplitude values of the selected electrodes 26, which are displayed in direct association with the corresponding electrode representations 26′, as well as with the controls 114, 116, and 118. The non-alphanumeric indicators can be, e.g., different colors, different color luminances, different patterns, different textures, different partially-filled objects, etc. These non-alphanumeric indicators provide a better visual than does the alphanumeric indicators for the user to determine the polarity and stimulation amplitude value of a selected electrode 26.

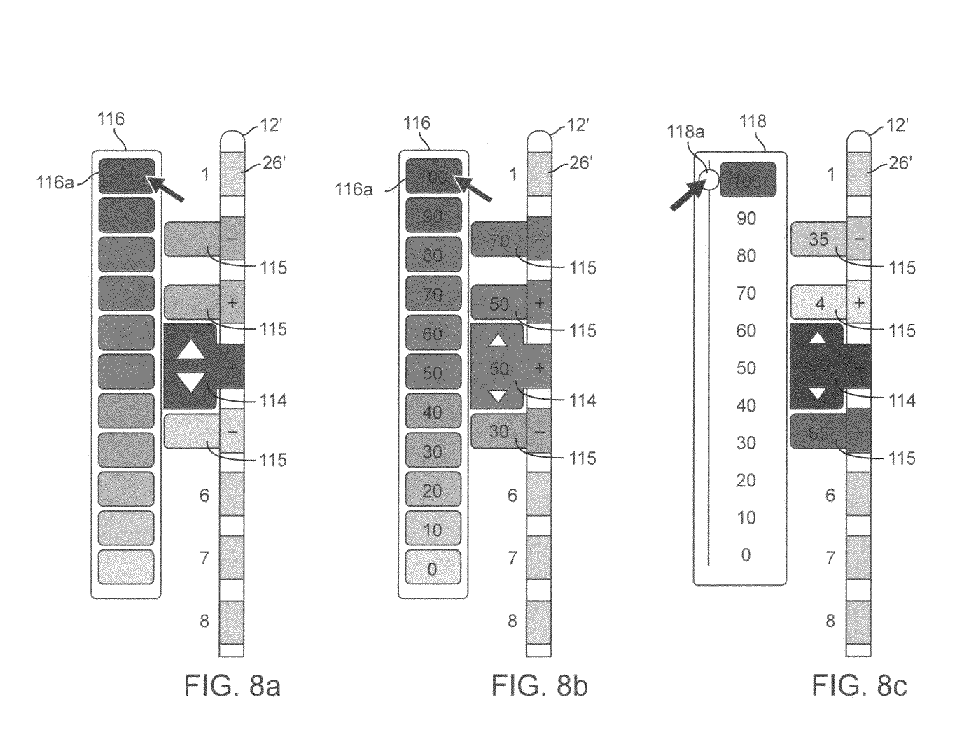

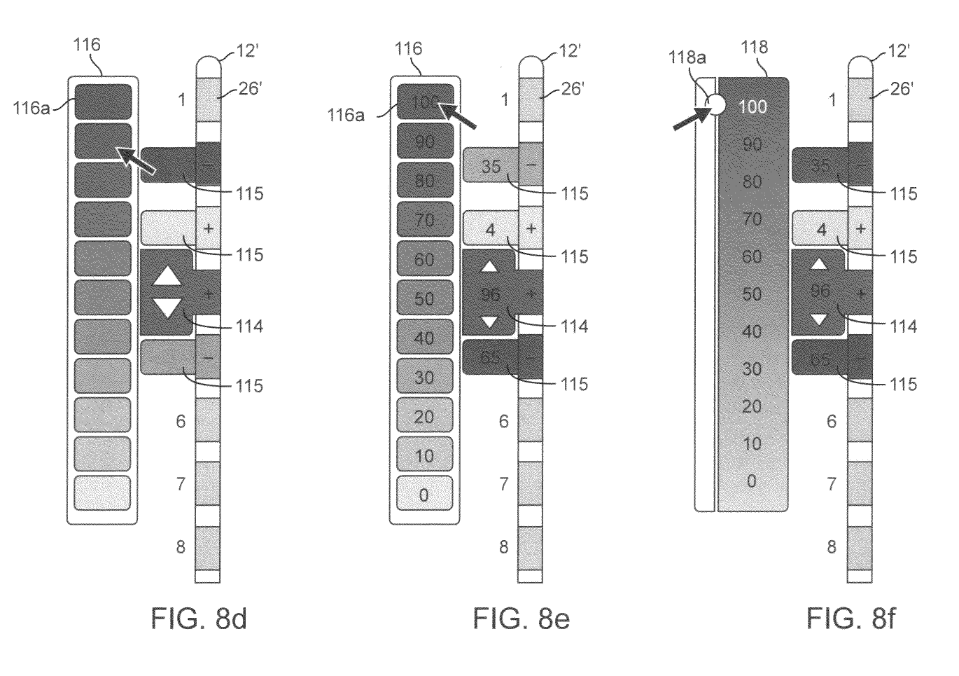

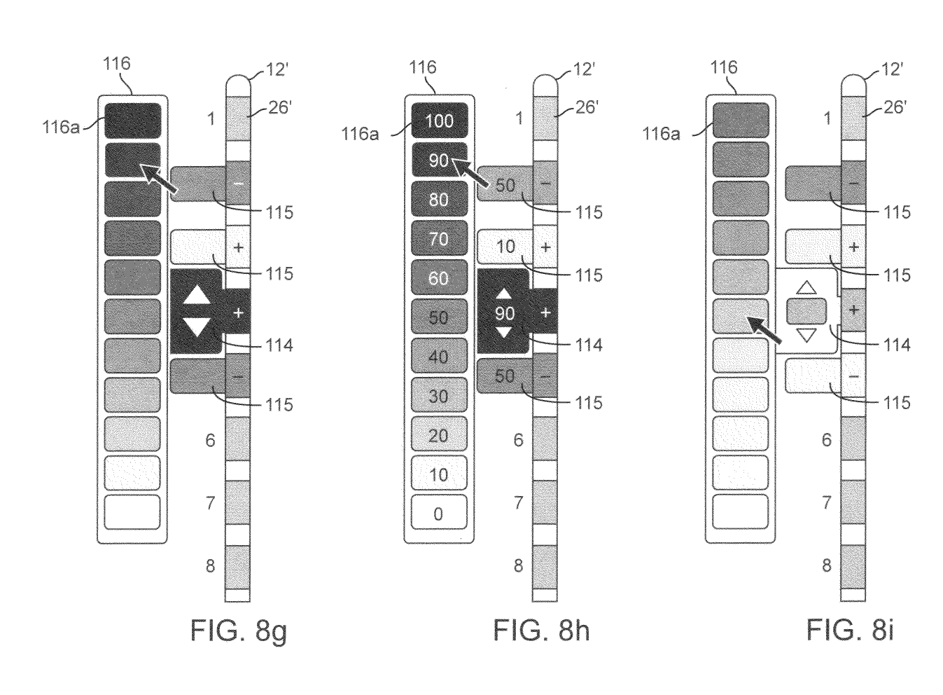

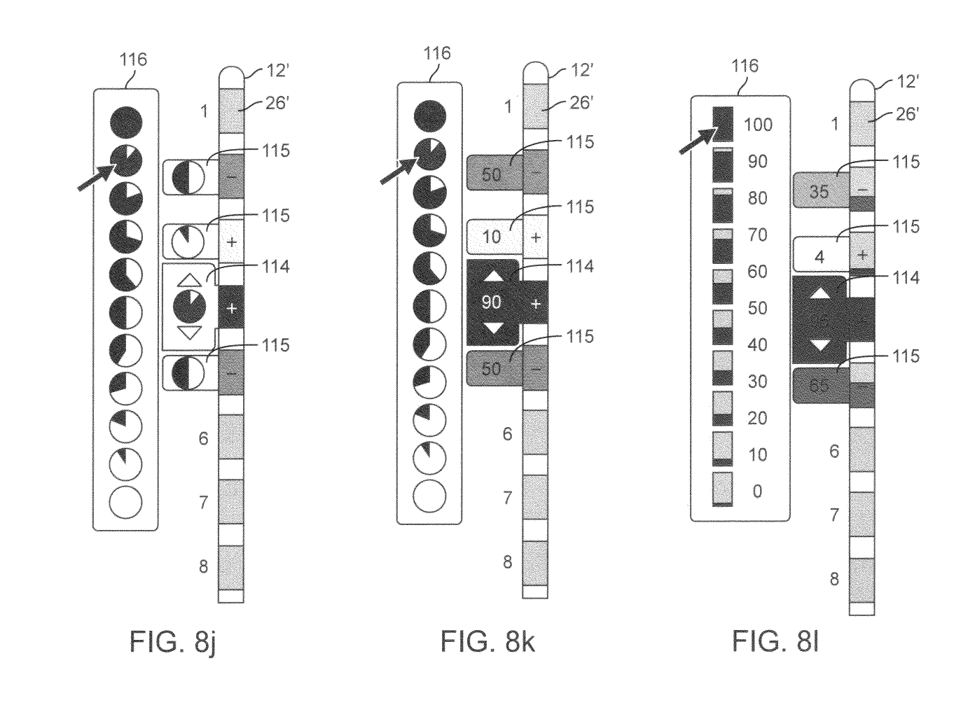

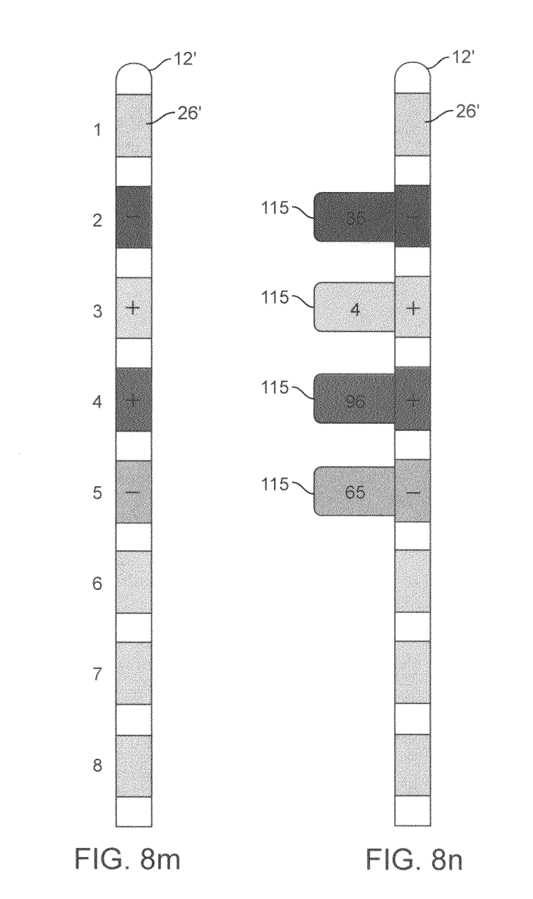

Referring now to FIGS. 8a-8n, different types of non-alphanumeric indicators will be illustrated and described.

In FIG. 8a, the alphanumeric indicators for the polarities take the form of pluses (“+”) and minuses (“−”) that are displayed within the selected electrode representations 26′. In the illustrated embodiment, two minuses are respectively displayed within the electrode representations 26′ corresponding to electrodes E2 and E5, and two pluses are respectively displayed within the electrode representations 26′ corresponding to electrodes E3 and E4, providing an indication to the user that electrodes E2 and E5 are programmed as cathodes and electrodes E3 and E4 are programmed as anodes.

In the example illustrated in FIG. 8a, there are no non-alphanumeric indicators for the polarities, at least none that distinguish anodes and cathodes. However, non-alphanumeric indicators for the stimulation amplitude values take the form of different luminance for a specific color hue displayed within the selected electrode representations 26′. Notably, the color hue may be defined in accordance with any graphical model, e.g., a 24-bit RGB triplet (8 bit value for red, 8 bit value for green, and 8 bit value for blue). For example, blue can be represented by the RBG triplet (0, 0, 255), red can be represented as (255, 0, 0), yellow can be represented as (255, 255, 0), cyan can be represented as (0, 255, 255), magenta can be represented as (255, 0, 255), and gray can be represented as (128, 128, 128).

In the embodiment of FIG. 8a, non-alphanumeric indicators for the stimulation amplitude values take the form of different luminance for a blue hue ranging from white (high luminance) (255, 255, 255), indicating a relatively low stimulation amplitude value, to a medium blue (medium luminance) (0, 0, 255), indicating a relatively high stimulation amplitude value. In the illustrated embodiment, a medium blue color is displayed within the electrode representation 26′ corresponding to electrode E2, indicating that it has a relatively high stimulation amplitude value, a medium-light blue color is displayed within the electrode representation 26′ corresponding to electrode E3, indicating that it has a relatively medium stimulation amplitude value, and a light blue color is displayed within the electrode representations 26′ corresponding to electrodes E4 and E5, indicating that they have relatively low stimulation amplitude values.

In addition to being displayed within the electrode representations 26′ themselves, the non-alphanumeric indicators for the stimulation amplitude values (i.e., the color luminance) are also displayed within flags 115 that are graphically coupled to the selected electrode representations 26′. The non-alphanumeric indicators for the stimulation amplitude values (i.e., the color luminance) are also displayed within the stimulation amplitude adjustment control 114 and the respective palette regions 116a of the graphical palette 116 to allow the user to more easily correlate the palette regions 116a with the desired stimulation amplitude values.

Although the embodiment illustrated in FIG. 8a does not display alphanumeric indicators for the stimulation amplitude values, it can be appreciated that numerical indicators can be displayed within the flags 115 and/or graphical palette 116. For example, as shown in FIG. 8b, numerical values of 70, 50, 50, and 30 are displayed within the flags 115 associated with the electrode representations 26′, indicating to the user that the stimulation amplitude values assigned to electrodes E2-E5 are 70%, 50%, 50% and 30%, respectively, and numerical values ranging from 0 to 100 in increments of 10 are displayed in the respective palette regions 116a, indicating to the user that the graphical palette 116 can be used to assign stimulation amplitude values ranging from 0% to 100% to a selected electrode 26 in increments of 10%.

In the embodiment of FIG. 8c, a stimulation amplitude slider 118 is used instead of a graphical palette 116. As the slider object 118a is moved, a different non-alphanumerical indicator (in this case, a different blue luminance) is displayed in the slider 118 as the slider object 118a moves adjacent to a different stimulation amplitude value in 10% increments. A different non-alphanumerical indicator (in this case, a different blue luminance) is displayed in the selected electrode representation 16′ in 1% increments, and thus may be a slightly different luminance than the indicator currently displayed in the slider 118.

The embodiment illustrated in FIG. 8d is similar to the embodiment illustrated in FIG. 8a, with the exception that it includes non-alphanumeric indicators for the polarities that take the form of different color hues displayed within the selected electrode representations 26′. In the illustrated embodiment, a blue color hue is displayed within the electrode representations 26′ corresponding to electrodes E2 and E5, and a red color hue is displayed within the electrode representations 26′ corresponding to electrodes E3 and E4, providing an additional indication to the user that a negative polarity is assigned to electrodes E2 and E5 and a positive polarity is assigned to electrodes E3 and E4.

As with the embodiment in FIG. 8a, the non-alphanumeric indicators for the stimulation amplitude values take the form of different color luminance displayed within the selected electrode representations 26′. In this case, luminance for the blue hue (for cathodes) ranges from white (high luminance) (255, 255, 255), indicating a relatively low stimulation amplitude value, to a medium blue (medium luminance) (0, 0, 255), indicating a relatively high stimulation amplitude value, and luminance for the red hue (for cathodes) ranges from white (high luminance) (255, 255, 255), indicating a relatively low stimulation amplitude value, to a medium red (medium luminance)(255, 0, 0), indicating a relatively high stimulation amplitude value.

In the illustrated embodiment, a medium blue color is displayed within the electrode representation 26′ corresponding to electrode E2, indicating that it has a relatively high cathodic stimulation amplitude value, a light blue color is displayed within the electrode representation 26′ corresponding to electrode E5, indicating that it has a relatively low cathodic stimulation amplitude value, a light red color is displayed within the electrode representation 26′ corresponding to electrode E3, indicating that it has a relatively low anodic stimulation amplitude value, and a medium red color is displayed within the electrode representation 26′ corresponding to electrode E4, indicating that it has a relatively high anodic stimulation amplitude value.

The color hue within the graphical palette 116 will vary depending upon the polarity of the currently selected electrode 26. In this case, the currently selected electrode 26 has a positive polarity (anodic), and therefore, the color hue within the graphical palette is red. If the currently selected electrode 26 has a negative polarity (cathodic), the color hue within the graphical palette will be blue.

Although the embodiment illustrated in FIG. 8d does not display alphanumeric indicators for the stimulation amplitude values, it can be appreciated that numerical indicators can be displayed within the flags 115 and/or graphical palette 116. For example, as shown in FIG. 8e, numerical values of 65 and 35 are displayed within the flags 115 associated with the electrode representations 26′ corresponding to electrodes E2 and E5, indicating to the user that the cathodic stimulation amplitude values assigned to electrodes E2 and E5 are 65% and 35%, respectively, and 4 and 96 are displayed within the flags 115 associated with the electrode representations 26′ corresponding to electrodes E3 and E4, indicating to the user that the anodic stimulation amplitude values assigned to electrodes E3 and E4 are 4% and 96%, respectively. Numerical values ranging from 0 to 100 in increments of 10 are displayed in the respective palette regions 116a, indicating to the user that the graphical palette 116 can be used to assign stimulation amplitude values ranging from 0% to 100% to a selected electrode 26 in increments of 10%.

In the embodiment of FIG. 8f, a stimulation amplitude slider 118 is used instead of a graphical palette 116. Unlike with the embodiment of FIG. 8c, a red hue has a graduated luminance that remains displayed within the slider 118 regardless of the relative position of the slider object 118a. Furthermore, the non-alphanumerical indicator (in this case, a different red luminance) is displayed in the selected electrode representation 16′ that exactly matches the red luminance in the slider 118 corresponding to the relative position of the slider object 118a.

Although the non-alphanumeric indicators in the previous embodiments are chromatic (i.e., not white, black, or gray), the non-alphanumeric indicators can be achromatic (i.e., any shade of gray, including white and black). For example, the embodiment of FIG. 8g displays non-alphanumeric indicators for the stimulation amplitude values in the form of different luminance for a grey hue ranging from white (high luminance) (255, 255, 255), indicating a relatively low stimulation amplitude value, to a black (low luminance) (0, 0, 0), indicating a relatively high stimulation amplitude value. In the illustrated embodiment, a medium grey is displayed within the electrode representations 26′ corresponding to electrodes E2 and E5, indicating that it has a relatively medium stimulation amplitude value, a light gray is displayed within the electrode representation 26′ corresponding to electrode E3, indicating that it has a relatively low stimulation amplitude value, a dark grey is displayed within the electrode representation 26′ corresponding to electrode E4, indicating it has a relatively high stimulation amplitude value.

In addition to being displayed within the electrode representations 26′ themselves, the non-alphanumeric indicators for the stimulation amplitude values (i.e., the color luminance) are also displayed within flags 115 that are graphically coupled to the selected electrode representations 26′. The non-alphanumeric indicators for the stimulation amplitude values (i.e., the color luminance) are also displayed within the stimulation amplitude adjustment control 114 and the respective palette regions 116a of the graphical palette 116 to allow the user to more easily correlate the palette regions 116a with the desired stimulation amplitude values.

Although the embodiment illustrated in FIG. 8g does not display alphanumeric indicators for the stimulation amplitude values, it can be appreciated that numerical indicators can be displayed within the flags 115 and/or graphical palette 116. For example, as shown in FIG. 8h, numerical values of 50, 10, 90, and 50 are displayed within the flags 115 associated with the electrode representations 26′, indicating to the user that the stimulation amplitude values assigned to electrodes E2-E5 are 50%, 10%, 90%, and 50%, respectively, and numerical values ranging from 0 to 100 in increments of 10 are displayed in the respective palette regions 116a, indicating to the user that the graphical palette 116 can be used to assign stimulation amplitude values ranging from 0% to 100% to a selected electrode 26 in increments of 10%.

The embodiment illustrated in FIG. 8i is similar to the embodiment illustrated in FIG. 8g, with the exception that, instead of different solid grey colors, different gray patterns or textures are used for the non-alphanumeric indicators. The patterns or textures range from a very dense pattern or texture, indicating a relatively high stimulation amplitude value, to no pattern or texture, indicating a relatively low stimulation amplitude value. In the illustrated embodiment, a relatively highly dense pattern or texture is displayed within the electrode representation 26′ corresponding to electrode E2, indicating that it has a relatively high stimulation amplitude value, a relatively low density pattern or texture is displayed within the electrode representation 26′ corresponding to electrode E3, indicating that it has a relatively low stimulation amplitude value, a relatively medium dense pattern or texture is displayed within the electrode representation 26′ corresponding to electrode E4, indicating that it has a relatively medium stimulation amplitude value, and a very low density pattern is displayed within the electrode representation 26′ corresponding to electrode E5, indicating that it has a very low stimulation amplitude value.

In addition to being displayed within the electrode representations 26′ themselves, the non-alphanumeric indicators for the stimulation amplitude values (i.e., the different patterns or textures) are also displayed within flags 115 that are graphically coupled to the selected electrode representations 26′. The non-alphanumeric indicators for the stimulation amplitude values (i.e., the different patterns or textures) are also displayed within the stimulation amplitude adjustment control 114 and the respective palette regions 116a of the graphical palette 116 to allow the user to more easily correlate the palette regions 116a with the desired stimulation amplitude values.

The embodiment illustrated in FIG. 8j is similar to the embodiments illustrated in FIGS. 8g and 8h, with the exception that, instead of different grey colors and patterns or textures, different partial-fill objects, and in particular pie-shaped objects, are used for the non-alphanumeric indicators of the stimulation amplitude values. The pie-shaped objects range from a totally full pie, indicating a relatively high stimulation amplitude value, to an empty pie, indicating a relatively low stimulation amplitude value.

In the illustrated embodiment, a half-full pie is displayed within the flag 115 graphically coupled to the electrode representations 26′ corresponding to electrodes E2 and E5, indicating that they have medium stimulation amplitude values, an almost empty pie is displayed within the flag 115 graphically coupled to the electrode representation 26′ corresponding to electrode E3, indicating that it has a relatively low stimulation amplitude value, and an almost full pie is displayed within the control element 114 graphically coupled to the electrode representation 26′ corresponding to electrode E4, indicating that it has a relatively high stimulation amplitude value. In addition to being displayed within the flags 115 and control element 114, the non-alphanumeric indicators for the stimulation amplitude values (i.e., the different filled pies) are also displayed within the graphical palette 116.

Although the embodiment illustrated in FIG. 8j does not display alphanumeric indicators for the stimulation amplitude values, it can be appreciated that numerical indicators can be displayed within the flags 115. For example, as shown in FIG. 8k, numerical values of 50, 10, 90, and 50 are displayed within the flags 115 associated with the electrode representations 26′, indicating to the user that the stimulation amplitude values assigned to electrodes E2-E5 are 50%, 10%, 90%, and 50%, respectively.

The embodiment illustrated in FIG. 81 is similar to the embodiment illustrated in FIG. 8f, with the exception that, instead of using different luminance, different partial-fill objects, and in particular bar-shaped objects, are used for the non-alphanumeric indicators of the stimulation amplitude values. The bar-shaped objects range from a totally full bar, indicating a relatively high stimulation amplitude value, to a nearly empty bar, indicating a relatively low stimulation amplitude value. Similar to the embodiment of FIG. 8f, a partially filled bar having a blue color hue is displayed within the electrode representations 26′ and flags 115 corresponding to electrodes E2 and E5, and a partially filed bar having a red color hue is displayed within the electrode representations 26′ and flags 115 corresponding to electrodes E3 and E4, providing an additional indication to the user that a negative polarity is assigned to electrodes E2 and E5 and a positive polarity is assigned to electrodes E3 and E4. The color hue within the graphical palette 116 will vary depending upon the polarity of the currently selected electrode 26. In this case, the currently selected electrode 26 has a positive polarity (anodic), and therefore, the color hue within the graphical palette is red. If the currently selected electrode 26 has a negative polarity (cathodic), the color hue within the graphical palette will be blue.

In the illustrated embodiment, an approximately third filled bar is displayed within the flag 115 graphically coupled to the electrode representation 26′ corresponding to electrode E2, indicating that it has a relatively low amplitude value, an almost empty bar is displayed within the flag 115 graphically coupled to the electrode representation 26′ corresponding to electrode E3, indicating that it has a very low stimulation amplitude value, an almost full bar is displayed within the control element 114 graphically coupled to the electrode representation 26′ corresponding to electrode E4, indicating that it has a very high stimulation amplitude value, an approximately two-thirds filled bar is displayed within the flag 115 graphically coupled to the electrode representation 26′ corresponding to electrode E5, indicating that it has a relatively high stimulation amplitude value. In addition to being displayed within the flags 115 and control element 114, the non-alphanumeric indicators for the stimulation amplitude values (i.e., the different filled bars) are also displayed within the graphical palette 116.

Once the polarities and stimulation amplitude values have been finally assigned to the electrodes 26, the lead representation 12′ and electrode representations 26′ may be displayed without the control element 114 and graphical palette 116 or slider 118. For example, after the polarities and stimulation amplitudes values have been assigned to the electrodes 26 in the embodiments illustrated in FIGS. 8d-8f, the lead representation 12′ and electrode representations 26′ may be displayed as shown in FIGS. 8m and 8n. In the embodiment of FIG. 8m, the display is very simplistic in that no flags 115, no control elements, and no alphanumerical indicators of the stimulation amplitude values are shown. In the embodiment of FIG. 8n, the alphanumerical indicators of the stimulation amplitude values are displayed within the flags 115.

Although the non-alphanumeric indicators are particularly useful when displayed as an indication of polarities and stimulation amplitude values for electrodes, it should be appreciated that non-alphanumeric indicators can be useful in certain circumstances to indicate other types of stimulation parameters, including pulse width and pulse rate. Furthermore, although the foregoing techniques have been described as being implemented in the CP 18, it should be noted that this technique may be alternatively or additionally implemented in the RC 16.

Although particular embodiments of the present inventions have been shown and described, it will be understood that it is not intended to limit the present inventions to the preferred embodiments, and it will be obvious to those skilled in the art that various changes and modifications may be made without departing from the spirit and scope of the present inventions. Thus, the present inventions are intended to cover alternatives, modifications, and equivalents, which may be included within the spirit and scope of the present inventions as defined by the claims.

What is claimed is:

1. An external control device for use with a neurostimulation system having a neurostimulation lead carrying a plurality of electrodes capable of conveying an electrical stimulation field into tissue in which the electrodes are implanted, comprising:
a user interface including one or more control elements and a display screen; and
a processor configured for individually assigning stimulation amplitude values for selected ones of the electrodes in response to actuations of the one or more control elements and for displaying on the display screen representations of the electrodes and a plurality of first non-alphanumeric indicators of the stimulation amplitude values in graphical association with the respective representations of the selected electrodes.

2. The external control device of claim 1, wherein the first non-alphanumeric indicators are displayed in direct graphical association with the respective representations of the selected electrodes.

3. The external control device of claim 1, wherein the first non-alphanumeric indicators are different colors for the respective stimulation amplitude values.

4. The external control device of claim 3, wherein the different colors comprise different luminance of the same color hue.

5. The external control device of claim 3, wherein the different colors are chromatic.

6. The external control device of claim 3, wherein the different colors are achromatic.

7. The external control device of claim 1, wherein the first non-alphanumeric indicators are different patterns or textures for the respective stimulation amplitude values.

8. The external control device of claim 1, wherein the first non-alphanumeric indicators are different partial fill objects for the respective stimulation amplitude values.

9. The external control device of claim 8, wherein the different partial fill objects are different partially field pie-shaped objects.

10. The external control device of claim 1, wherein the processor is further configured for individually assigning polarities for the selected ones of the electrodes in response to actuations of the one or more control elements, and for displaying second non-alphanumeric indicators of the polarities in direct graphical association with the respective representations of the selected electrodes.

11. The external control device of claim 10, wherein the second non-alphanumeric indicators are different color hues for the respective polarities.

12. The external control device of claim 11, wherein a first of the different color hues is blue for one of a positive polarity and a negative polarity, and a second of the different color hues is red for the other of the positive polarity and the negative polarity.

13. The external control device of claim 1, wherein the first non-alphanumeric indicators are graphically coupled to the representations of the electrodes corresponding to the selected electrodes.

14. The external control device of claim 13, wherein the representations of the electrodes respectively take the form of closed geometric figures, and the first non-alphanumeric indicators are displayed in the closed geometric figures corresponding to the selected electrodes.

15. The external control device of claim 1, wherein the processor is configured for displaying the one or more control elements as one or more graphical icons on the display screen.

16. The external control device of claim 15, wherein the one or more graphical icons comprises a graphical control icon graphically coupled to the representation of one of the selected electrodes, wherein the processor is configured for increasing or decreasing the stimulation amplitude value for the selected electrode in response to actuation of the graphical control icon.

17. The external control device of claim 16, wherein the processor is configured for additionally displaying the first non-alphanumeric indicator of the increased or decreased stimulation amplitude value within the graphical control icon.

18. The external control device of claim 15, wherein the one or more graphical icons comprises a graphical palette having a plurality of regions that allows a user to discretely select one of a plurality of stimulation amplitude values for one of the selected electrodes, and wherein the processor is configured for assigning the one stimulation amplitude value, when selected by the user, to the selected electrode.

19. The external control device of claim 18, wherein the processor is configured for additionally displaying the first non-alphanumeric indicators respectively within the regions of the graphical palette.

20. The external control device of claim 14, wherein the one or more graphical icons comprises a graphical slider that allows a user to continuously select one of a plurality of stimulation amplitude values for one of the selected electrodes, and wherein the processor is configured for assigning the one stimulation amplitude value, when selected by the user, to the selected electrode.

21. The external control device of claim 20, wherein the processor is configured for additionally displaying the first non-alphanumeric indicators respectively within graphical slider.
